(19) United States
(12) Patent Application Publication
(10) Pub. No.: US 2025/0051380 A1
(43) Pub. Date: Feb. 13, 2025

(54) NEW STERICALLY ACTIVATED CHELATING RUTHENIUM COMPLEXES, METHOD OF THEIR PREPARATION AND THEIR USE IN OLEFIN METATHESIS REACTIONS

(71) Applicant: APEIRON SYNTHESIS S.A., Wroclaw (PL)

(72) Inventors: Krzysztof Skowerski, Jablonowo Pomorskie (PL); Konrad Kurcbach, Brzeziny (PL); Michal Chwalba, Siechnice (PL); Patryk Krajczy, Glogowek (PL); Mateusz Krzysztof Cybulski, Warszawa (PL)

(21) Appl. No.: 18/722,570

(22) PCT Filed: Dec. 22, 2022
(86) PCT No.: PCT/PL2022/050096

§ 371 Date: Jun. 20, 2024

(30) Foreign Application Priority Data
Dec. 23, 2021 (PL) P.439977

(51) Int. Cl. C07F 15/00; B01J 31/22; C07C 6/04

(52) U.S. Cl. CPC C07F 15/0046; B01J 31/2208; B01J 31/226; B01J 31/2295; C07C 6/04; B01J 2231/543; B01J 2531/0238; B01J 2531/821; C07C 2531/12

(57) ABSTRACT

The subject of the invention are new sterically activated chelating ruthenium complexes with the formula 1a, easy to obtain by efficient chemical reactions. The invention also concerns the method of obtaining and using ruthenium complexes with formula 1a as precatalysts and/or catalysts in a wide spectrum of known olefin metathesis reactions.

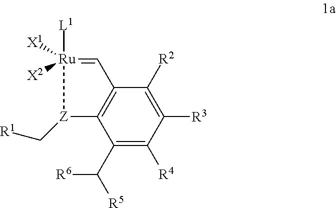

The subject of the invention are new, sterically activated chelating ruthenium complexes, useful as catalysts and/or (pre)catalysts for olefin metathesis reactions, method for their preparation from commercially available substrates and their use in a wide spectrum of known olefin metathesis reactions. This invention is used as a tool in broadly understood organic synthesis.

In recent years, great progress has been made in the use of olefin metathesis in organic synthesis [R. H. Grubbs (Editor), A. G. Wenzel (Editor), D. J. O'Leary (Editor), E. Khosravi (Editor), Handbook of Olefin Metathesis, 2nd edition, 3 volumes 2015, John Wiley & Sons, Inc., 1608 pages]. In the state of the art, many homogeneous olefin metathesis catalysts based on ruthenium are known, which have both high activity in various types of metathesis reactions and high tolerance to functional groups in the substrate/product. By combining these characteristics, metathesis catalysts are essential in modern organic synthesis and industry. The most widely described in the literature (pre)catalysts are Grubbs, Hoveyda and indenylidene complexes, containing in their structure the N-heterocyclic carbene ligand (NHC). Recently, Bertrand-type catalysts with a cycloalkylamine carbene ligand (CAAC) have been extensively studied [Grubbs et al. Chem. Rev. 2010, 110, 1746-1787; WO2017055945A1]. In other cases, most of the structures of olefin metathesis catalysts (illustrated by the following formulas) are derived from the above-mentioned ruthenium complexes.

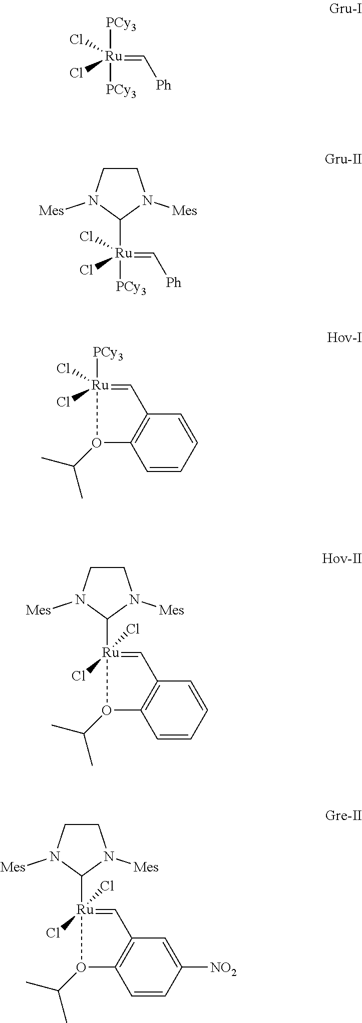

There are many examples of industrial applications of ruthenium complexes, in which it is beneficial to use (pre)catalysts with a high initiation rate. A representative example is the use of a Gre-II catalyst in a macrocyclization reaction [Farina, V, Shu, C., Zeng, X, Wei, X, Han, Z., Yee, N. K., Senanayake, C. H., Org. Process Res. Dev., 2009, 13, 250-254]. The use of Gre-II in this process allowed to significantly reduce the amount of catalyst used, as well as reduce the volume of solvent in relation to the conditions of the process in which the Hov-I catalyst was used. Increasing the initiation rate of the Gre-II catalyst was achieved by inserting an electron-acceptor nitro group into the benzylidene ligand [WO2004035596A1]. The nitro substituent causes a decrease in electron density on the ether oxygen atom, which chelates the ruthenium atom in the structure of the catalyst complex. As a consequence, the chelating effect of Ru—O is weakened, thanks to which the Gre-II complex is a rapid initiator of metathesis reactions [K. Grela and A. Kajetanowicz, Angew. Chem. Int. Ed. 2021, 60, https://doi.org/10.1002/anie.202008150]. It is worth emphasizing that the activity of Hoveyda catalysts containing substituents in the benzylidene ring is well correlated with the value of Hammett constants of the aromatic ring substituents. By selecting appropriate electron-acceptor substituents (which extract electrons from the aromatic ring), Hoveyda catalysts can be activated, while electron-donor substituents (supplying electrons to the aromatic ring) deactivate catalysts, which allows obtaining slow or dormant catalysts, which are of great importance in ROMP polymerization reactions (metathetic polymerization of cycloolefins with ring opening). In this way, planning the activity of Hoveyda catalysts by modifying the electron cloud density of the benzylidene ring by engineering substituents (electron-acceptor or electron-donor) is relatively easy to determine and implement.

An alternative, less popular, strategy for activation of the chelating ruthenium complexes promoting olefin metathesis reaction, consisting in the synthesis of ligands containing steric hindrance in their structure, is also known in the state of the art. These hindrances repel the ligand that chelates the central ruthenium atom, so that the catalyst is active at temperatures lower than is possible for similar catalysts without such steric hindrances. The oldest examples of sterically activated catalysts are the Ble-1 and Ble-2 structures revealed in 2002 [H. Wakamatsu and S. Blechert, Angew. Chem. Int. Ed. 2002, 41, 794-796, H. Wakamatsu and S. Blechert, Angew. Chem. Int. Ed. 2002, 41, 2509-2511]. Nevertheless, in the case of ligand synthesis for the Ble-2 complex, the difficult to scale-up and expensive Wittig reaction is used (in which large amounts of waste in the form of O═PPh₃ are formed), while the alternative synthesis path with Claisen rearrangement does not allow to obtain a sufficiently pure product (low selectivity).

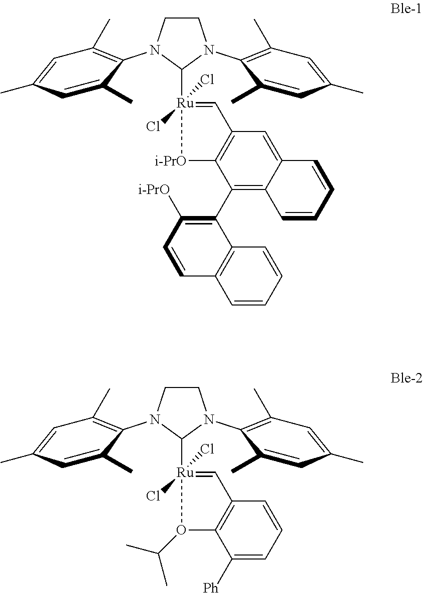

In the state of the art, catalyst structures containing a chelating oxygen atom substituted by other substituents are also known, such as: (i) ester substituent X revealed in 2006 [K. Grela, and others, J. Am. Chem. Soc. 2006, 128, 13652-13653; K. Grela et al., Organometallics 2011, 30, 4144-4158]; (ii) benzyl substituent Y; (iii) 2,3-dihydrobenzofuran V substituent revealed in 2007 [K. Grela et al., Adv. Synth. Catal. 2007, 349, 193-203]; (iv) phenyl substituent Z [H. Plenio et al., Adv. Synth. Catal. 2013, 355, 439-447]. The activated X, Y, V and Z complexes showed an increase in activity compared to the Hov-H complex but were significantly slower initiators than Ble-2. It is interesting that the effects responsible for the increase in the activity of complexes X, Y, V and Z are not always easy to indicate (steric or electron). For example, the responsibility for the high activity of the X catalyst can be attributed to the electron-acceptor effects of the ester group, as well as the additional chelation of the ruthenium atom by carbonyl oxygen and the increased size of the substituent at the ether oxygen atom.

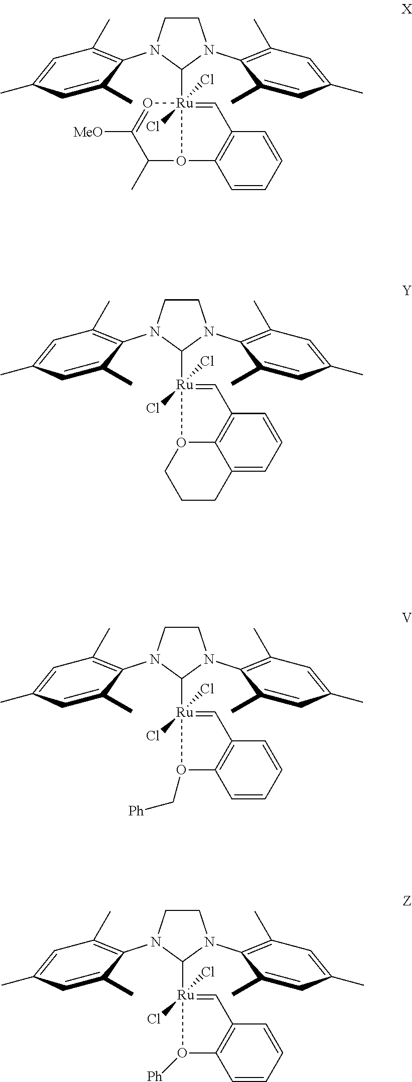

In the state of the art, ruthenium complexes are also known, which contain sterically developed substituents at the chelating oxygen atom. For example, tridentate pincer Karl catalysts containing a carboxylate group in the benzylidene fragment [Grela et al., Angew. Chem. Int. Ed. 2007, 46, 7206-7209], are chemically activated with Brønsted acids, e.g. HCl. In the development of this solution, an analogous catalyst with more bulky substituents such as iso-propyl instead of methyl, in the structure of the Kar-2 complex was presented [Grela et al., Eur. J. Inorg. Chem. 2012, 1477-1484]. In a standard study aimed at comparing the activity of catalysts in the metathetic ring closure reaction of RCM in the case of dimethyl diallylmalonate, it was observed that carboxylate catalysts (Kar-1 and Kar-2) after their activation with HCl are more active than Hov-II, which can be explained by the electron effect of the carboxyl group. However, when comparing both carboxylate complexes with each other, an increased activity of the complex containing a more bulky isopropyl substituent (Kar-2) is observed compared to the complex containing the methyl substituent (Kar-1).

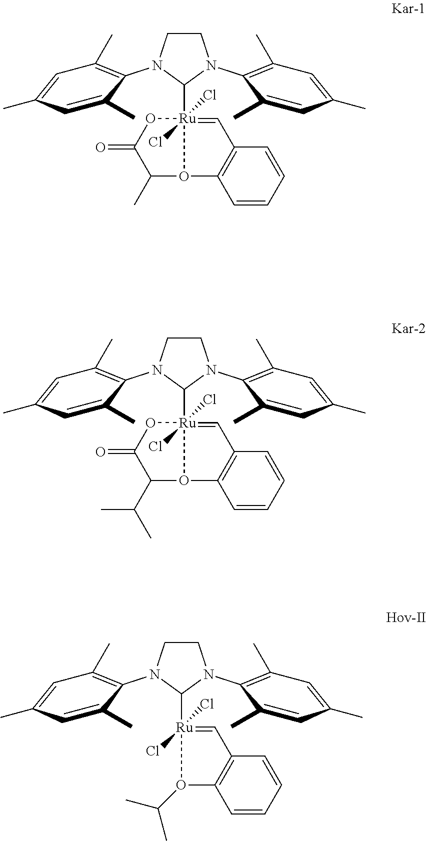

In the state of the art, chelating ruthenium complexes containing an oxygen atom substituted with spatially expanded substituents such as neopentyl (Neo) and adamantyl (Ada-1 and Ada-2), as well as a smaller substituent—cyclic cyclopropyl (C3) are also known. Studies on the rate of initiation of these ruthenium complexes were revealed together with their reactivity profiles in model reactions [Grubbs et al., J. Am. Chem. Soc. 2015, 137, 5782-5792; Grubbs et al., J. Org. Chem. 2015, 80, 4213-4220]. The results of the study of activity in model reactions turned out to be interesting, because two conformational isomers of the adamantyl substituent are characterized by different activity in the RCM reaction, i.e. Ada-1 is more active than Ada-2. On the other hand, all catalysts are more active than Hov-II, with their activity changing in the following growing series: Hov-II<C3<Ada2<Neo<Ada-2

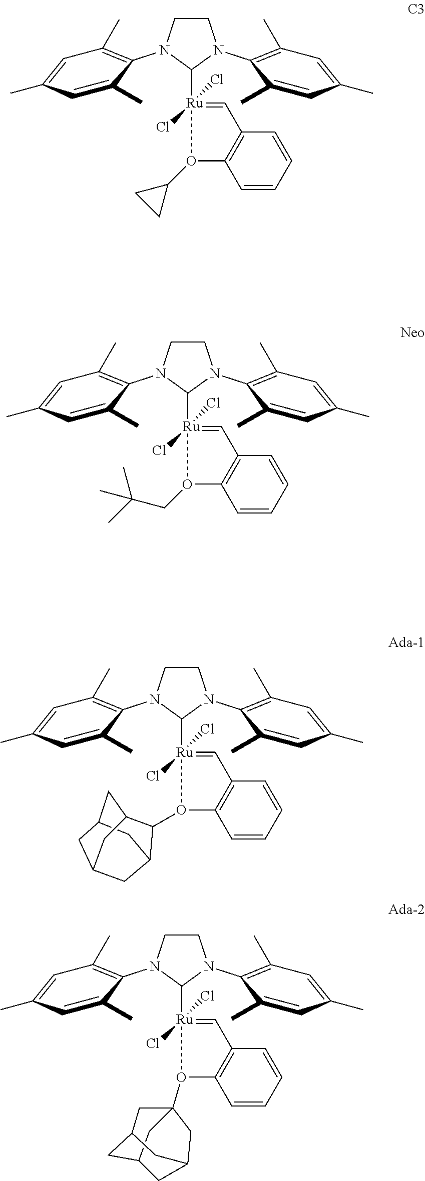

A little later, Grela et al., revealed the results of their research on the activity of ruthenium complexes substituted with various cyclic derivatives at the chelating oxygen atom [Grela et al., Eur. J. Inorg. Chem. 2018, 3675-3685]. In the model RCM reaction carried out at room temperature, the activity of ruthenium complexes in the initial phase of the reaction changed in the following non-intuitive series of activities: C5≈C3>>Hov-II>C4>C6>C7. Thus, it was observed that the connection between the activity of the chelating ruthenium complex and its spatial structure is much more difficult than it might initially seem. Unlike electron activation of Hoveyda catalysts, steric activation of catalyst structures is not possible to predict from the structure of a ruthenium catalyst.

It should be noted that Grubbs et al. [J. Am. Chem. Soc., 2015, 137, 5782-5792] showed that the initiation rate of ruthenium catalysts (C3, Ada 2, Neo, Ada-2) was higher than its non-activated predecessor (Hov-H), but at the same time this activity was lower than the difficult to obtain sterically activated Ble-2 complex.

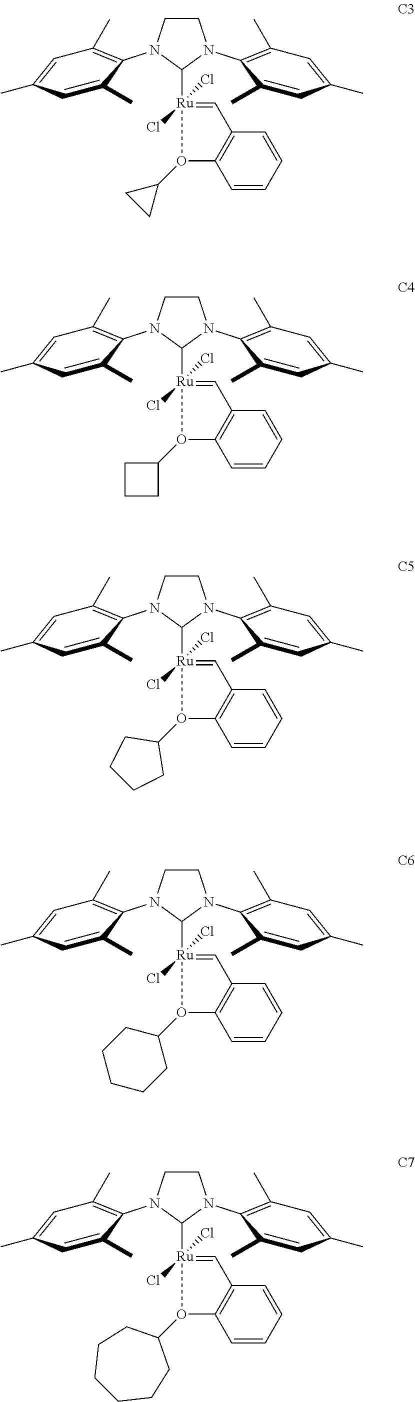

In conclusion, it is currently not possible to predict the catalytic activity of a sterically modified ruthenium complex (as opposed to electron activation) without obtaining the planned structure and studying its catalytic activity in model reactions.

In the literature, chelating benzylidene complexes of the Hoveyda-type are known, containing in the structure pincer-type dithiocatechol ligand as an anionic ligand [WO2014201300A1, and A. Hoveyda et al., J. Am. Chem. Soc., 2013, 135, 10258-10261].

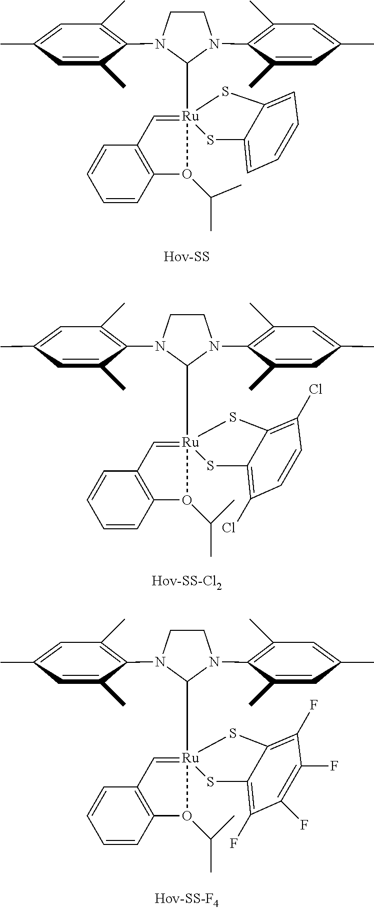

Chelating catalysts containing various modifications of dithiocatechol ligand (Hov-SS, Hov-SS—Cl₂, Hov-SS—F₄) are characterized by high stereoretentivity—that is, the ability to transfer the original configuration of substrates to products within the double bond C═C. It is worth noting that the reactions were carried out at room temperature with high catalyst loading, with a long exposure of the catalyst to substrates/products (up to 12 hours, THF, room temperature, 1 mol %). At the same time, the first literature reports described the activity of ruthenium dithiocatechol catalysts in ROMP reactions and rarely used ROM/CM reactions, hence it was difficult to compare the activity of new catalysts with those previously reported.

From the point of view of industrial application, the easy availability of the catalyst is of great importance. This is related, among other things, to the trouble-free synthesis of its precursors (ligands) on a large-scale scale. Despite the high activity of the (pre) catalyst Gre-II, the synthesis of its precursor—benzylidene ligand is multi-stage and associated with a number of technological problems, difficult to solve on a large-scale preparation. In the case of the precursor of the Gre-II complex, some stages require strict reaction conditions (fuming nitric acid or temperature >200° C.), and the efficiency of the intermediate products is low, due to the low selectivity of the nitration reaction (costly purification of the aromatic ring isomers [ortho, para]) or the formation of by-products in the reaction of the Claisen rearrangement. From the point of view of industrial application, it is therefore desirable to develop a class of catalysts easily available on an industrial scale, as well as with a wide range of applications and high chemical tolerance to the presence of various functional groups in substrates.

The aim of this invention was to develop a new class of sterically activated ruthenium catalysts, with an activity significantly exceeding the activity of the Gre-IT complex (electron-activated) while maintaining stability parameters. An extremely important parameter from the industrial point of view is that simple ligands should be used in its synthesis and those should be obtained as a result of efficient reactions, using easily available substrates.

In addition, the aim of the invention was also to develop new ruthenium complexes with similar activity in olefin metathesis reactions to Ble-2 (sterically activated), which are at the same time easier to synthesize on a large scale (synthesis reactions are efficient and selective, in addition, they do not generate large amounts of waste and by-products).

Unexpectedly, it was found that (pre)catalysts—ruthenium complexes according to the invention presented by formula 1—show high activity in olefin metathesis reactions at low temperatures, without additional chemical activation. In addition, in the structure of ruthenium complexes described by formula 1 in the benzylidene substituent there is one secondary carbon atom, unlike in the well-known ruthenium complexes Ble-1 and Ble-2, which have two tertiary carbon atoms in their structure. All this makes the synthesis path leading to the ruthenium complexes described in Formula 1 much more efficient and is based on simple chemical reactions.

The high activity of ruthenium complexes having a sterically modified benzylidene ligand (described in Formula 1) is a beneficial factor compensating for the decrease in the activity of (pre)ruthenium catalysts, which have anionic dithiol ligands (stereoretentive catalysts).

In the first aspect of the invention, a ruthenium complex of a structure represented by formula 1a

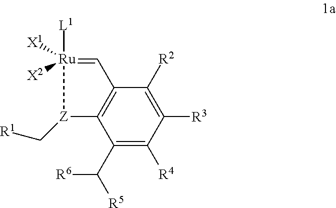

wherein:
L¹ is a neutral ligand selected from N-heterocyclic carbene (NHC) and cyclic alkylaminocarbene (CAAC);
X¹ and X², are independently selected from halogen atom, groups —OR′, —SR′, —O(C═O)R′ or —O(SO2)R′, in which R′ is alkyl C₁-C₁₂, cycloalkyl C₃-C₁₂ or aryl C₆-C₂₀, which are optionally substituted by at least one alkyl C₁-C₁₂, perfluoroalkyl C₁-C₁₂, alkoxyl C₁-C₁₂, aryloxyl C₆-C₂₄ or halogen atom, where X¹ and X² may be combined to form a bidentate ligand, or R¹ may be combined with L¹ to form a poly-dentate ligand;
Z is selected from an O atom, an S atom or an NR″ group in which R″ is alkyl C₁-C₁₂, cycloalkyl C3-C₁₂, alkenyl C2-C₁₂ or aryl C₆-C₂₀, which are optionally substituted by at least one alkyl C₁-C₁₂, perfluoroalkyl C₁-C₁₂, alkoxyl C₁-C₁₂, aryloxyl C₆-C₂₄, aryloxyl C₆-C₂₄ or halogen atom;
R¹ is selected from a hydrogen atom, alkyl C₁-C₂₅, alkoxyl C₅-C₂₀, aryl C₅-C₂₀, aryloxyl C₅-C₂₄, group —COOR′″, —CONR′″₂, —COR′″, —CON(OR′″)(R′″) or a halogen atom, with R′″ being alkyl C₁-C₁₂, cycloalkyl C₃-C₁₂, alkenyl C₂-C₁₂ or aryl C₆-C₂₀, which are optionally substituted by at least one alkyl C₁-C₁₂, perfluoroalkyl C₁-C₁₂, alkoxyl C₁-C₁₂, aryloxyl C₆-C₂₄ or an atom halogen;
R², R³ and R⁴, are independently selected from hydrogen atom, alkyl C₁-C₂₅, alkoxyl C₁-C₂₅, alkenyl C₂-C₂₅, —OR^{a}, —SR^{a}, —S(O)R^{a}, —S⁺R a₂, —SO₂R^{a}, —NR^{a}₂, —N⁺R^{a}₃, —NO₂, —CN, —P(O)(OR^{a})₂, —P(O)R^{a}(OR^{a}), —P(OR^{a})₂, —PR^{a}₂, —P(O)R^{a}₂, —P⁺R^{a}₃, —COOH, —COOR^{a}, —CONR^{a}₂, —NR^{a}C(O)R^{a}, —CHO, —COR^{a},
   in which R^{a} is alkyl C₁-C₅, perfluoroalkyl C₁-C₅, aryl C₆-C₂₄, aralkil C₇-C₂₄, perfluoroaryl C₅-C₂₄, and substituents R² R³ and R⁴ can be combined with each other to form a substituted or unsubstituted C₄-C₁₀ or polycyclic C₄-C₁₂ system;
R⁵ and R⁶, are independently selected from alkyl C₁-C₂₅, alkoxyl C₅-C₂₀, aryl C₅-C₂₀, aryloxyl C₅-C₂₄, alkenyl C₂-C₁₂, heteroaryl C₆-C₂₀ or heteroaryloxyl C₅-C₂₄, which are optionally substituted by at least one alkyl C₁-C₁₂, perfluoroalkyl C₁-C₁₂, or alternatively R⁵ and R⁶ are combined to form a substituted or unsubstituted C₄-C₁₀ or polycyclic C₄-C₁₂ system.

Preferably, the complex in which the neutral ligand L¹ is selected from 2a, 2b or 2c:

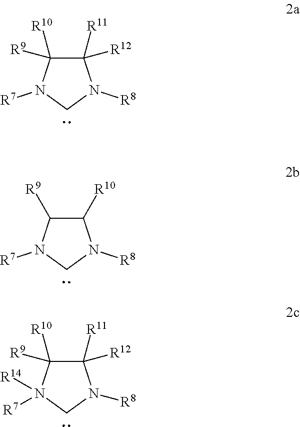

wherein:
each substituent R⁷ and R⁸ are independently selected from C₁-C₁₂ alkyl, C₃-C₁₂ cycloalkyl, C₅-C₂₀ aryl or C₅-C₂₀ heteroaryl, which can be substituted by one and/or more substituents selected from a hydrogen atom, C₁-C₁₂ alkyl, C₁-C₁₂ perhaloalkyl, C₁-C₁₂ alkoxyl, C₅-C₂₄ aryloxil, C₅-C₂₀ heteroaryloxil and halogen atom;
each substituent R⁹, R¹⁰, R¹¹, R¹², R¹³ and R¹⁴ independently of the others means a hydrogen atom, C₁-C₁₂ alkyl, C₃-C₁₂ cycloalkyl, C₅-C₂₀ aryl or C₅-C₂₀ heteroaryl, which may be substituted by at least one substituent selected from a group composed of C₁-C₁₂ alkyl, C₁-C₁₂ perhaloalkyl, C₁-C₁₂ alkoxyl and halogen atom, where substituents R⁹, R¹⁰, R¹¹, R¹², R¹³ and R¹⁴ can optionally be combined with each other to form a C₄-C₁₀ cyclic system or a C₄-C₁₂ polycyclic system.

Preferably, the complex according to the invention, is a complex wherein:
X¹ and X² are independently an anionic ligand selected from chloride and iodide;
Z is selected from an oxygen atom or a sulfur atom;
R¹ is aryl C₅-C₂₀;
R², R³ and R⁴ independently denote a hydrogen atom, alkyl C₁-C₂₅, aryl C₆-C₂₄ or aralkil C₇-C₂₄;
R⁵ and R⁶ are independently selected from alkyl C₁-C₂₅, aryl C₅-C₂₀, heteroaryl C₆-C₂₀ or heteroaryloxyl C₅-C₂₄.

Preferably, the complex according to the invention, is a complex wherein:
X¹ and X² are independently an anionic ligand selected from chloride and iodide;
Z is an oxygen atom;
R¹ is alkyl C₁-C₂₅ or aryl C₅-C₂₀;
R², R³ and R⁴ are independently selected from hydrogen atom, alkyl C₁-C₂₅, alkoxyl C₁-C₂₅, alkenyl C₂-C₂₅, aryl C₆-C₂₄ or aralkil C₇-C₂₄;
R⁵ and R⁶, independently of each other, denote alkyl C₁-C₂₅, alkoxyl C₅-C₂₀, aryl C₅-C₂₀, aryloxyl C₅-C₂₄, alkenyl C₂-C₁₂, heteroaryl C₆-C₂₀, or heteroaryloxyl C₅-C₂₄.

Preferably, the complex according to the invention, is a complex of formula 1b

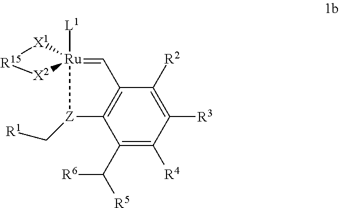

wherein:
L¹, R¹, R², R³, R⁴, R⁵ and R⁶ are as defined for a compound with general formula 1a, X¹ and X² are independently selected from —O—, —S—, —Se—, —OC(O)—, —OC(S)—, —SC(O)—, —SC(S)—, —P(R^{x})—, —P(O)(R^{x})—, or —N(R^{x})—;
R¹⁵ is the —B^{x}-group, the bifunctional aliphatic group C₁-C₂₀ or the heteroaliphatic group C₁-C₂₀, in which from 0 to 6 methylene units are independently replaced by —O—, —S—, —N(R′)—, —C(O)—, —OC(O)—, —C(O)O—, —OC(O)O—, —C(S)—, —OC(S)—, —SC(O)—, —SC(S)—, —C(O)—, —S(O)—, —S(O)₂—, —OS(O)₂—, —N(R′)C(O)—, —C(O)N(R′)—, —N(R′)C(O)O—, —OC(O)N(R′)—, —N(R′)C(O)N(R′)—, —P(R^{x})—, —P(O)(R^{x})— or -Cy¹-,
wherein each -Cy¹- is:
   a bifunctional unicyclic ring selected from benzene, C₃-C₈ saturated or partially unsaturated cycloalkene, C₅-C₆ heteroaryl having from 1 to 4 heteroatoms independently selected from nitrogen, oxygen or sulfur atoms, or C₃-C₈ saturated or partially unsaturated heterocycloalkene having from 1 to 4 heteroatoms independently selected from nitrogen, oxygen or sulphur atoms, or
   bifunctional bicyclic or polycyclic ring selected from C₈-C₁₄ aryl, C₇-C₁₄ saturated or partially unsaturated cycloalkene, C₈-C₁₄ heteroaryl having from 1 to 5 heteroatoms independently selected from nitrogen, oxygen or sulfur atoms, or C₇-C₁₄ saturated or partially unsaturated heterocycloalkene having between 1 and 5 heteroatoms independently selected from nitrogen, oxygen or sulphur atoms, wherein —B^{x}— is selected from the group comprising following structures:

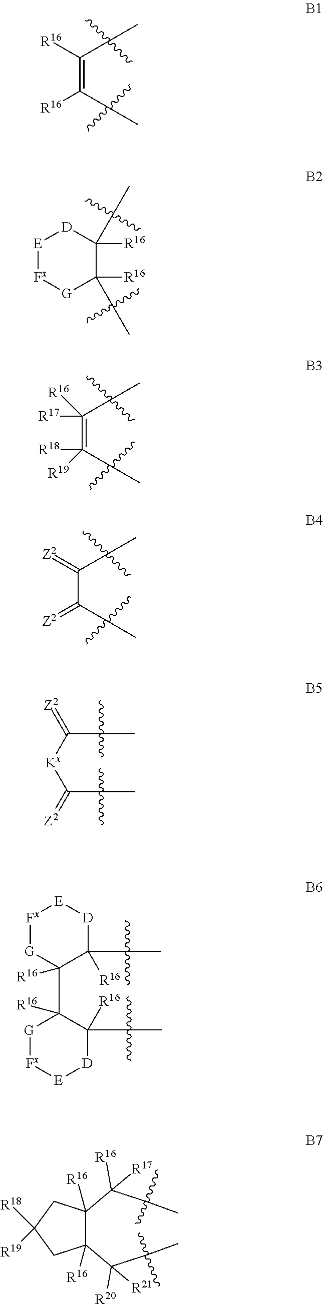

in which:
each Z² are independently selected from ═C(R^{x})₂, ═O, ═S, ═Se, ═N(R^{x}), ═P(R^{x}), ═C═O, ═C═S, ═S═O or ═Se═O;
each of the D, E, F^{x}, G are independently selected from —N(R¹⁶)—, —C(R¹⁶)₂—, —S—, —O—, —Se—, —P(R¹⁶)—, —C(O)—, —S(O)— or —Se(O)—;
each of R¹⁶, R¹⁷, R¹⁸, R¹⁹, R²⁰ and R²¹ are independently selected from R^{x}, or
one of R¹⁶, R¹⁷, R¹⁸, R¹⁹, R²⁰ or R²¹ independently of the others is connected to another substituent R¹⁶, R¹⁷, R¹⁸, R¹⁹, R²⁰ or R²¹, with the same atom that forms the group ═C(R^{x})₂, =N(R^{x}), ═P(R^{x}), ═O, ═S, ═Se; or
one of R¹⁶, R¹⁷, R¹⁸, R¹⁹, R²⁰ or R²¹, independently of the others, is connected to another substituent R¹⁶, R¹⁷, R¹⁸, R¹⁹, R²⁰ or R²¹, to an adjacent atom forming a double bond C═C; or
one of the R¹⁶, R¹⁷, R¹⁸, R¹⁹, R²⁰ or R²¹ is independently connected to another substituent R¹⁶, R¹⁷, R¹⁸, R¹⁹, R²⁰ or R²¹ and their atoms form C₃-C₁₀ saturated cycloalkanes, partially unsaturated cycloalkenes, aryls or heteroaryls containing from 1 to 4 heteroatoms selected from sulphur, nitrogen, oxygen and selenium atoms; and
Kx is a bifunctional aliphatic group C₁-C₂₀ or heteroaliphatic group C₁-C₂₀, in which from 0 to 6 methylene units are optionally replaced by —O—, —S—, —N(R′)—, —C(O)—, —OC(O)—, —C(O)O—, —OC(O)O—, —C(S)—, —OC(S)—, —SC(O)—, —SC(S)—, —C(O)—, —S(O)—, —S(O)₂—, —OS(O)₂—, —N(R′)C(O)—, —C(O)N(R′)—, —N(R′)C(O)O—, —OC(O)N(R′)—, —N(R′)C(O)N(R′)—, —P(R^{x})—, —P(O)(R^{x})— or -Cy¹-;
each R′ means R, —C(O)R, —C(O)N(R)₂, —C(O)OR, —SO₂R, —SO₂N(R)₂, P(O)(OR)₂ or —OR; and
each R^{x} is a halogen atom, —R, —CN, —C(O)N(R′)², —C(O)R, —C(O)OR, —OR, —OC(O)R, —OC(O)OR, —OC(O)N(R′)₂, —OSi(R)₃, —N(R′)₂, —N+(R′)₃, —NR′C(O)R, —NR′C(O)OR, —NR′C(O)N(R′)₂, —NR′SO₂N(R′)₂, —NR′OR, —NO₂, —Si(R)₃, —P(R)₂, —P(O)(R)₂, —P(O)(OR)₂, —SR, —SC(O)R, —S(O)R, —SO₂R, —SO₃R, —SO₂N(R′)₂ or —SeR;
each R is a hydrogen atom or a group selected from group C₁-C₂₀ aliphatic, group C₁-C₂₀ heteroaliphatic, group C₁-C₂₀ aromatic, C₃-C₇-membered a bicyclic saturated ring, partially unsaturated or aromatic, C₅-C₆-membered a heteroaromatic ring which has from 1 to 4 heteroatoms independently selected from nitrogen, oxygen and sulfur atoms, C₃-C₇ membered saturated or partially unsaturated heterocyclolyphatic ring having from 1 to 3 heteroatoms independently selected from nitrogen, oxygen and sulfur atoms, C₇-C₁₀ a membered bicyclic saturated or partially unsaturated heterocycloaliphatic ring having from 1 to 3 heteroatoms independently selected from nitrogen, oxygen and sulphur atoms, C₈-C₁₀ membered bicyclic heteroaromatic ring which has between 1 and 5 heteroatoms independently selected from nitrogen, oxygen and sulphur atoms; or
the two substituents R are joined together by atoms belonging to them and form C₃-C₁₀ saturated cycloalkane, partially unsaturated cykoalken, aryl or heteroaryl containing from 1 to 4 heteroatoms selected from sulfur, nitrogen, oxygen and selenium atoms;
or two or more substituents from R¹, R², R³, R⁴, R⁵, R⁶, X¹, X², R⁷, R⁸, R⁸, R¹⁰, R¹¹, R¹², R¹³, R¹⁴, R¹⁵, R¹⁶, R¹⁷, R¹⁸, R¹⁹, R²⁰ and R²¹, are combined together to form a bifunctional or multifunctional ligand;
where each of R¹, R², R³, R⁴, R⁵, R⁶, X¹ and X² can be optionally and independently connected to a solid carrier or chemical tracer.

Preferably, the complex according to the invention, is selected from the group represented by the structures 1a-Cl₂, 1a-I₂, 1a-S₂, 1b-Cl₂, 1b-I₂, 1b-S₂, 1c-Cl₂, 1c-I₂, 1c-S₂, 1d-S₂, 1e-S₂:

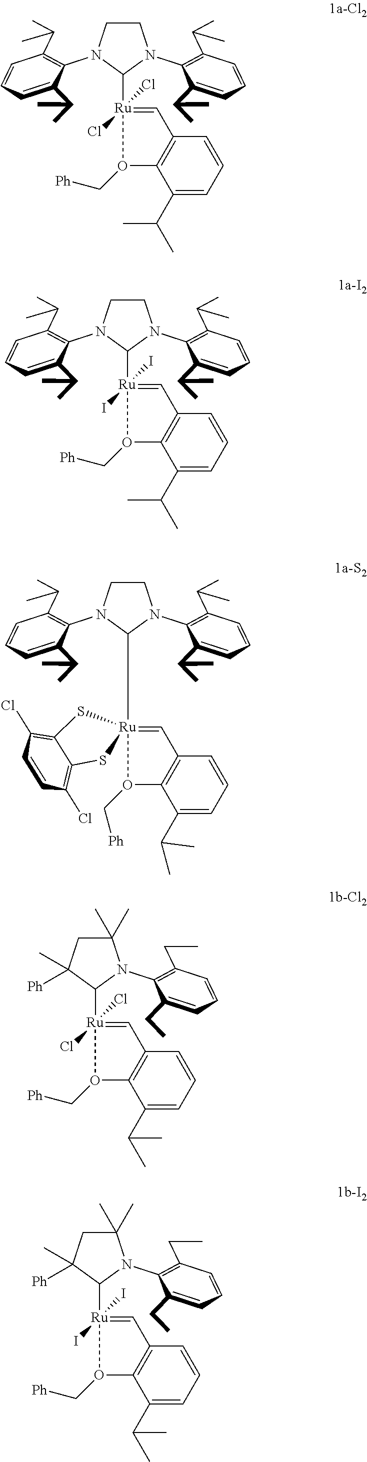

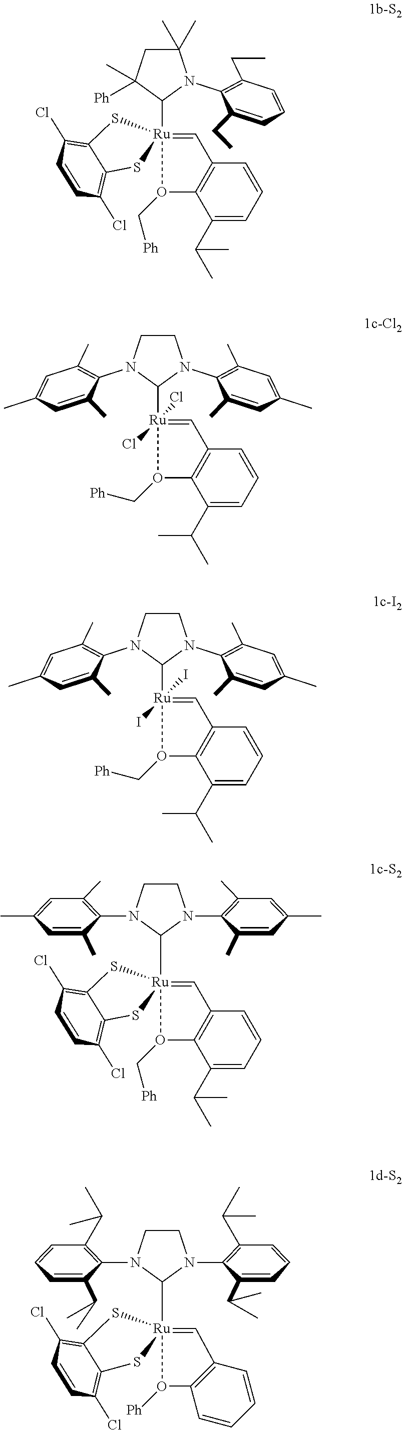

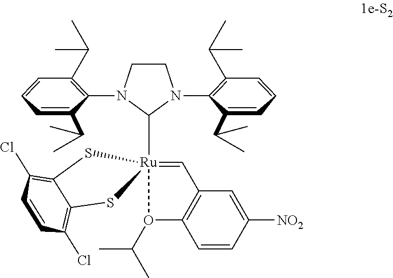

Another aspect of the invention is an ether derivative of styrene of general formula 3a

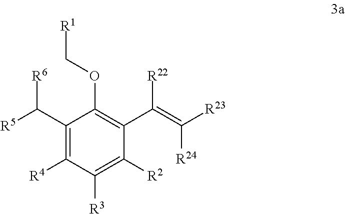

wherein:
R¹ denotes aryl C₅-C₂₀, aryloxyl C₅-C₂₄, group —COOR′″, —CONR′″₂, —COR′″, —CON(OR′″)(R′″) or halogen atom, with R′″ denoting alkyl C₁-C₁₂, cycloalkyl C₃-C₁₂, alkenyl C₂-C₁₂ or aryl C₆-C₂₀ which are optionally substituted by at least one alkyl C₁-C₁₂, perfluoroalkyl C₁-C₁₂, alkoxyl C₁-C₁₂, aryloxyl C₆-C₂₄ or a halogen atom;
R², R³ and R⁴ are independently selected from hydrogen atom, alkyl C₁-C₂₅, alkoxyl C₁-C₂₅ or alkenyl C₂-C₂₅, —OR^{a}, —SR^{a}, —S(O)R^{a}, —S⁺R^{a}₂, —SO₂R^{a}, —NR^{a}₂, —N⁺R^{a}₃, —NO₂, —CN, —P(O)(OR^{a})₂, —P(O)R^{a}(OR^{a}), —P(OR^{a})₂, —PR^{a}₂, —P(O)R^{a}₂, —P⁺R^{a}₃, —COOH, —COOR^{a}, —CONR^{a}₂, —NR^{a}C(O)R^{a}, —CHO, —COR^{a},
   In which R^{a} is alkyl C₁-C₅, perfluoroalkyl C₁-C₅, aryl C₆-C₂₄, aralkil C₇-C₂₄ or perfluoroaryl C₅-C₂₄, where substituents R², R³ and R⁴ are combined with each other to form a substituted or unsubstituted C₄-C₁₀ or polycyclic C₄-C₁₂ system;
R⁵ and R⁶ independently of each other denote alkyl C₁-C₂₅, alkoxyl C₅-C₂₀, aryl C₅-C₂₀, aryloxyl C₅-C₂₄, alkenyl C₂-C₁₂, heteroaryl C₆-C₂₀ or heteroaryloxyl C₅-C₂₄, which are optionally substituted by at least one alkyl C₁-C₁₂ or perfluoroalkyl C₁-C₁₂, or R⁵ and R⁶ are combined with the formation of a substituted or unsubstituted C₄-C₁₀ or polycyclic C₄-C₁₂ system;
R²², R²³ and R²⁴, independently of each other, denote a hydrogen atom, alkyl C₁-C₂₅, cycloalkyl C₁-C₂₅, alkoxyl C₅-C₂₀, aryl C₅-C₂₀, aryloxyl C₅-C₂₄, group —COOR′″, —CONR″″₂, —COR′″, —CON(OR′″)(R′″) or halogen atom, with R′″ being alkyl C₁-C₁₂, cycloalkyl C₃-C₁₂, alkenyl C₂-C₁₂ or aryl C₆-C₂₀, which are optionally substituted by at least one alkyl C₁-C₁₂, perfluoroalkyl C₁-C₁₂, alkoxyl C₁-C₁₂, aryloxyl C₆-C₂₄ or halogen atom.

Preferably, the ether derivative of styrene has a structure defined by formula 3b

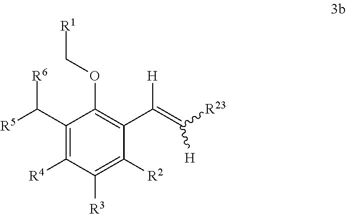

wherein:
R¹ denotes aryl C₅-C₂₀;
R², R³ and R⁴, independently of each other, denote a hydrogen atom, alkyl C₁-C₂₅, alkoxyl C₁-C₂₅ or alkenyl C₂-C₂₅, —OR^{a}, —SR^{a}, —S(O)R^{a}, —S⁺R^{a}₂, —SO₂R^{a}, —NR^{a}₂, —N⁺R^{a}₃, —NO₂, —CN, —P(O)(OR^{a})₂, —P(O)R^{a}(OR^{a}), —P(OR^{a})₂, —PR^{a}₂, —P(O)R^{a}₂, —P⁺R^{a}₃, —COOH, —COOR^{a}, —CONR^{a}₂, —NR^{a}C(O)R^{a}, —CHO, —COR^{a},
In which R^{a} is alkyl C₁-C₅, perfluoroalkyl C₁-C₅, aryl C₆-C₂₄, aralkyl C₇-C₂₄ or perfluoroaryl C₅-C₂₄, where substituents R², R³ and R⁴ are combined with each other to produce a substituted or unsubstituted C₄-C₁₀ or polycyclic C₄-C₁₂ system;
R⁵ and R⁶ independently of each other denote alkyl C₁-C₂₅ or cycloalkyl C₁-C₂₅,
R²³ is alkyl C₁-C₂₅, cycloalkyl C₁-C₂₅, alkoxyl C₅-C₂₀, aryl C₅-C₂₀, aryloxyl C₅-C₂₄.

Preferably, the ether derivative of styrene has a structure defined by formula 3c

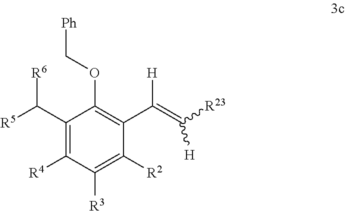

wherein:
R², R³ and R⁴, independently of each other, denote a hydrogen atom, alkyl C₁-C₂₅, alkoxyl C₁-C₂₅ or alkenyl C₂-C₂₅, —OR^{a}, —SR^{a}, —S(O)R^{a}, —S⁺R^{a}₂, —SO₂R^{a}, —NR^{a}₂, —N⁺R^{a}₃, —NO₂, —CN, —P(O)(OR^{a})₂, —P(O)R^{a}(OR^{a}), —P(OR^{a})₂, —PR^{a}₂, —P(O)R^{a}₂, —P⁺R^{a}₃, —COOH, —COOR^{a}, —CONR^{a}₂, —NR^{a}C(O)R^{a}, —CHO, —COR^{a},
In which R^{a} is alkyl C₁-C₅, perfluoroalkyl C₁-C₅, aryl C₆-C₂₄, aralkyl C₇-C₂₄ or perfluoroaryl C₅-C₂₄, where substituents R², R³ and R⁴ are combined with each other to produce a substituted or unsubstituted C₄-C₁₀ or polycyclic C₄-C₁₂ system;
R⁵ and R⁶ independently of each other denote alkyl alkyl C₁-C₂₅;
R²³ is alkyl C₁-C₂₅, cycloalkyl C₁-C₂₅.

Preferably, the ether derivative of styrene has a structure defined by formula 3d

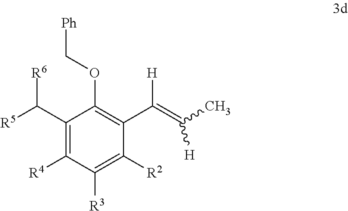

wherein:
R², R³ or R⁴, independently of each other, denote a hydrogen atom, alkyl C₁-C₂₅, alkoxyl C₁-C₂₅ or alkenyl C₂-C₂₅, —OR^{a}, —SR^{a}, —S(O)R^{a}, —S⁺R^{a}₂, —SO₂R^{a}, —NR^{a}₂, —N⁺R^{a}₃, —NO₂, —CN, —P(O)(OR^{a})₂, —P(O)R^{a}(OR^{a}), —P(OR^{a})₂, —PR^{a}₂, —P(O)R^{a}₂, —P⁺R^{a}₃, —COOH, —COOR^{a}, —CONR^{a}₂, —NR^{a}C(O)R^{a}, —CHO, —COR^{a},
In which R^{a} is alkyl C₁-C₅, perfluoroalkyl C₁-C₅, aryl C₆-C₂₄, aralkyl C₇-C₂₄ or perfluoroaryl C₅-C₂₄, where substituents R², R³ and R⁴ are combined with each other to produce a substituted or unsubstituted C₄-C₁₀ or polycyclic C₄-C₁₂ system;
R⁵ and R⁶ independently of each other denote alkyl alkyl C₁-C₂₅.

Preferably, the ether derivative of styrene has a structure defined by formula 3e

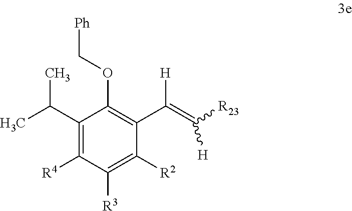

wherein:
R², R³ and R⁴, independently of each other, denote a hydrogen atom, alkyl C₁-C₂₅, alkoxyl C₁-C₂₅ or alkenyl C₂-C₂₅, —OR^{a}, —SR^{a}, —S(O)R^{a}, —S⁺R^{a}₂, —SO₂R^{a}, —NR^{a}₂, —N⁺R^{a}₃, —NO₂, —CN, —P(O)(OR^{a})₂, —P(O)R^{a}(OR^{a}), —P(OR^{a})₂, —PR^{a}₂, —P(O)R^{a}₂, —P⁺R^{a}₃, —COOH, —COOR^{a}, —CONR^{a}₂, —NR^{a}C(O)R^{a}, —CHO, —COR^{a},
In which R^{a} is alkyl C₁-C₅, perfluoroalkyl C₁-C₅, aryl C₆-C₂₄, aralkyl C₇-C₂₄ or perfluoroaryl C₅-C₂₄, where substituents R², R³ and R⁴ are combined with each other to produce a substituted or unsubstituted C₄-C₁₀ or polycyclic C₄-C₁₂ system;
R²³ is, alkyl C₁-C₂₅, cycloalkyl C₁-C₂₅.

Another aspect of the invention is a method for preparing the compound of general formula 1a,

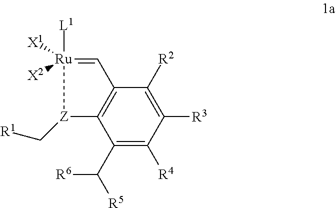

wherein L¹, X¹, X², R¹, R², R³, R⁴, R⁵ and R⁶ are as defined above for the compound of formula 1a, wherein the process is characterized in that it is alkylidene ruthenium complex of formula 4a

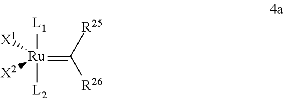

wherein:
L¹ is a group with the formula 2a, 2b or 2c:

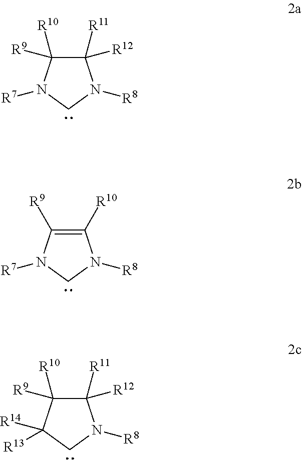

wherein R⁷, R⁸, R⁹, R¹⁰, R¹³ and R¹⁴ are as defined for the formulas 2a, 2b and 2c above,
L² is a neutral ligand selected from the group of O═S(R′)₂ or P(R′)₃, where each R′ independently of the others is alkyl C₁-C₁₂, cycloalkyl C₃-C₁₂, aryl C₅-C₂₀, aralkil C₇-C₂₄, perfluoroaryl C₅-C₂₄, or 5-12 membered heteroaryl;
X¹, X² are independently an anionic ligand selected from among halide anions, —CN, —SCN, —OR′, —SR′, —O(C═O)R′, —O(SO₂)R′ and —OSi(R′)₃, in which R′ is alkyl C₁-C₁₂, cycloalkyl C₃-C₁₂, alkenyl C₂-C₁₂ or aryl C₅-C₂₀ which is optionally substituted with at least one C₁-C₁₂ alkyl, perfluoroalkyl C₁-C₁₂, alkoxyl C₁-C₁₂, aryloxyl C₅-C₂₄, heteroaryloxyl C₅-C₂₀ or a halogen atom;
R¹¹, R¹² independently of each other denote the hydrogen atom, the halogen atom, alkyl C₁-C₂₅, perfluoroalkyl C₁-C₂₅, alkene C₂-C₂₅, cycloalkyl C₃-C₇, alkenyl C₂-C₂₅, cycloalkenyl C₃-C₂₅, alkyl C₂-C₂₅, cycloalkinyl C₃-C₂₅, alkoxyl C₁-C₂₅, aryloxyl C₅-C₂₄, heteroaryloxyl C₅-C₂₀, aryl C₅-C₂₄, heteroaryl C₅-C₂₀, aralkil C₇-C₂₄, perfluoroaryl C₅-C₂₄, 3-12 membered heterocycle;
wherein the substituents R¹¹ and R¹² are optionally connected to each other to form a ring selected from cycloalkyl C₃-C₇, cycloalkenyl C₃-C₂₅, cycloalkinyl C₃-C₂₅, aryl C₅-C₂₄, heteroaryl C₅-C₂₀, perfluoroaryl C₅-C₂₄, 3-12 member heterocycle, which is optionally substituted independently with at least one substituent selected from among a hydrogen atom, a halogen atom, alkyl C₁-C₂₅, perfluoroalkyl C₁-C₂₅, alkene C₂-C₂₅, cycloalkyl C₃-C₇, alkenyl C₂-C₂₅, cycloalkenyl C₃-C₂₅, alkyl C₂-C₂₅, cycloalkyl C₃-C₂₅, alkoxyl C₁-C₂₅, aryloxyl C₅-C₂₄, heteroaryloxyl C₅-C₂₀, aryl C₅-C₂₄, heteroaryl C₅-C₂₀, aralkil C₇-C₂₄, perfluoroaryl C₅-C₂₄ and 3-12 membered heterocycle;
reacts with compound of general formula 3a

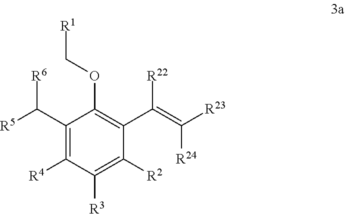

wherein R¹, R², R³, R⁴, R⁵, R⁶, R²², R²³ and R²⁴ are as defined above for the compound of general formula 3a.

Preferably, a method for preparing the compound of general formula 1b

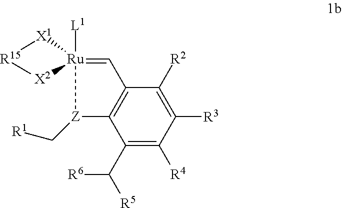

wherein:
L¹, R¹, R², R³, R⁴, R⁵ and R⁶ are as defined above for formula 1a, wherein the method is characterized in that ruthenium alkylidene complex of formula 1a

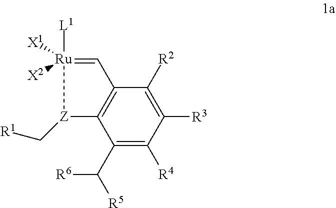

wherein L¹, X¹, X², R¹, R², R³, R⁴, R⁵ and R⁶ are as defined above for the compound of formula 1a,
contacts with a zinc complex compound with a formula of 5-B^{x}

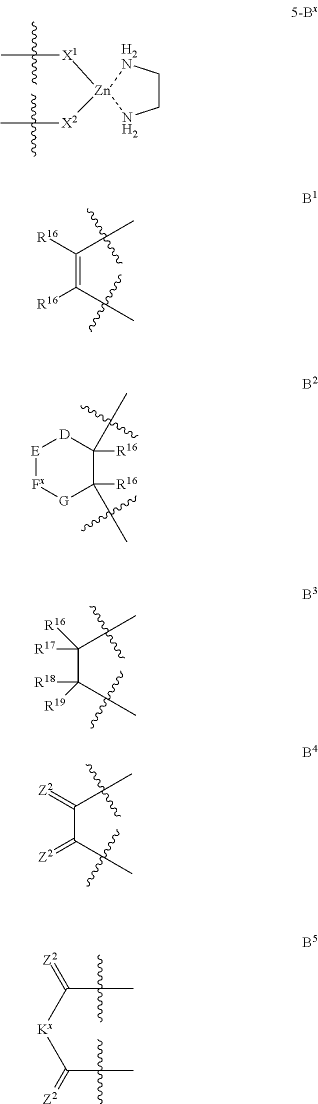

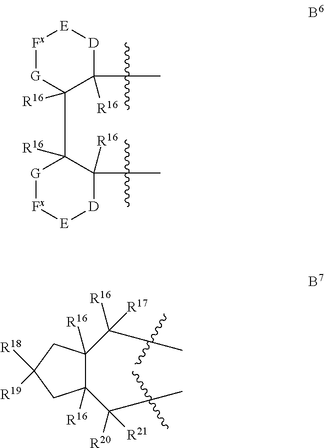

wherein X¹, X², R¹⁶, R¹⁷, R¹⁸, R¹⁹, R²⁰, R²¹, D, E, F^{x}, G, Z², K^{x} are as defined above for general formulas 5-B^{x}, B¹, B², B³, B⁴, B⁵, B⁶ and B⁷.

Another aspect of the invention is a use of a compound with formula 1a according to any of claims 1-6 as a precatalyst and/or catalyst in an olefin metathesis reactions, especially in a ring closing metathesis (RCM), a homometatesis, a cross metathesis (CM), an ethenolysis, an isomerization, a diastereoselective ring rearrangement metathesis (DRRM) reaction, an alken-alkin metathesis (en-yn) or a ring-opening metathesis polymerization (ROMP) reaction.

Preferably, the reaction is carried out in an organic solvent, selected from toluene, benzene, mesitylene, dichloromethane, ethyl acetate, methyl acetate, tertbutyl-methyl ether, cyclopentyl-methyl ether.

Preferably, the reaction is carried out in a solvent-free system Preferably, the reaction is carried out at a temperature of 20 to 150° C.

Preferably, the reaction is carried out in a time from 5 minutes to 24 hours.

Preferably, compound 1a is used in an amount of not more than 0.1% molar.

Preferably, compound 1a is added to the reaction mixture in solid and/or continuously by using vacuum pump as a solution.

Preferably, the gaseous by-product of the reaction selected from ethylene, propylene and butylene is actively removed from the reaction mixture by inert gas barbotage or by the vacuum

In this description, the terms used have the following meanings. The undefined terms in this document have meanings that are given and understood by a specialist in the field in light of the best knowledge available, this disclosure and the context of the description of the patent application. Unless otherwise stated, this description uses the following conventions of chemical terms that have indicated meanings such as those given in the definitions below:

As used in this description, the term ‘halogen atom’ means an element selected from a group of fluorine, chlorine, bromine, and iodine.

The term “carbene” means a molecule containing an inert carbon atom with a valence number of two and two unpaired valence electrons. The term “carbene” also includes carbene analogues, in which the carbon atom is replaced by another chemical element such as boron, silicon, germanium, tin, lead, nitrogen, phosphorus, sulfur, selenium, tellurium.

The term ‘alkyl’ refers to a saturated, linear, or branched hydrocarbon substituent with an indicated number of carbon atoms. Examples of an alkyl substituent are -methyl, -ethyl, -n-propyl, -n-butyl, -n-pentyl, -n-hexyl, -n-heptyl, -n-octyl, -n-nonyl, and -n-decile. Representative branched —(C1-C10)alkyls include -isopropyl, -sec-butyl, -isobutyl, -tert-butyl, -isopentyl, -neopentyl, -1-methylbutyl, -2-methylbutyl, -3-methylbutyl, -1,1-dimethylpropyl, -1,2-dimethylpropyl, -1-methylpentyl, -2-methylpentil, -3-methylpentil, -4-methylpentil, -1-ethylbutyl, -2-ethylbutyl, -3-ethylbutyl, -1,1-dimethylbutyl, -1,2-dimethylbutyI, 1,3-dimethylbutyl, -2,2-dimethylbutyl, -2,3-dimethylbutyl, -3,3-dimethyl-butyl, -1-methylhexyl, 2-methylhexyl, -3-methylhexyl, -4-methylhexyl, -5-methylhexyl, -1,2-dime-tipopentyl, -1,3-dimethylpentil, -1,2-dimethylhexyl, -1,3-dimethylhexyl, -3,3-dimethylhexyl, 1,2-di-methylheptyl, -1,3-dimethylheptyl, and -3,3-dimethylheptyl and the like.

The term “alkoxyl” refers to an alkyl substituent as defined above attached to the structure of the main compound via an oxygen atom.

The term “perfluoroalkyl” means an alkyl group as defined above in which all hydrogen atoms have been replaced by the same or different halogen atoms.

The term “cycloalkyl” refers to a saturated mono- or polycyclic hydrocarbon substituent with an indicated number of carbon atoms. Examples of cycloalkyl substituents are -cyclopropyl, -cyclobutyl, -cyclopentyl, -cyclohexyl, -cycloheptyl, cyclooctyl, -cyclocononyl, -cyclodecyl and the like.

The term ‘alkenyl’ refers to a saturated, linear, or branched non-cyclic hydrocarbon substituent with an indicated carbon number and containing at least one carbon-carbon double bond. Examples of an alkenyl substituent are -vinyl, -allil, -1-butenyl, -2-butenyl, -isobutylenyl, -1-pentenyl, -2-pentenyl, -3-methyl-1-butenyl, -2-methyl-2-butenyl, -2,3-di-methyl-2-butenyl, -1-hexenyl, -2-hexenyl, -3-hexenyl, -1-heptenyl, -2-heptenyl, -3-octenyl, -2-octenyl, -3-octenyl, -1-nonenyl, -2-nonenyl, -3-nonenyl, -1-decenyl, -2-decenyl, -3-decenyl and the like.

The term “aryl” refers to an aromatic mono- or polycyclic hydrocarbon substituent with an indicated number of carbon atoms. Examples of an aryl substituent are phenyl, -tolyl, -xylil, -naphthyl, -2,4,6-trimethylphenyl, -2-fluorophenyl, -4-fluorophenyl, -2,4,6-trifluorophenyl, -2,6-difluorophenyl, -4-nitrophenyl and the like.

The term “aralkil” refers to an alkyl substituent as defined above, substituted by at least one aryl as defined above. Examples of an aralkil substituent are benzyl, -diphenylmethyl, -triphenylmethyl and the like.

The term “heteroaryl” refers to an aromatic mono- or polycyclic hydrocarbon substituent with an indicated number of carbon atoms in which at least one carbon atom has been replaced by a heteroatom selected from among the O, N and S atoms. Examples of a heteroaryl substituent are -furyl, -tienyl, -imidazole, -oxazole, -thiazolil, -isoxazolil, -oxadiazolil, -thiadiazole, -tetrazolil, -pyridyl, -pyrimidyl, -triazineyl, -indolil, -benzo[b]furyl, -benzo[b]thienyl, -indazolil, -benzoimidazolil, -azaindolil, quinolil, isoquinolyl, carbazolil and the like.

The term “heterocycle” refers to a saturated or partially unsaturated, mono- or polycyclic hydrocarbon substituent, with an indicated number of carbon atoms, in which at least one carbon atom has been replaced by a heteroatom selected from among the O, N and S atoms. Examples of a heterocyclic substituent are furyl, thiophenyl, pyroryl, oxazolil, imidazole, thiazolil, isoxazolil, pyrazolinyl, pyrrolidinyl, pyrrolidinyl, hydantoinyl, oxiranyl, oxetalanyl, tetrahydrofuranyl, tetrahydrothiophenyl, quinolinel, isoquinolinyl, chromonyl, coumarinyl, indolil, indolisinyl, benzo[b]furanyl, benzo[b]thiophenyl, indazolil, purinil, 4H-quinolysine, isoquinolyl, quinolyl, phthalazinel, naphtharidinyl, carbazolil, β-carbolinyl and the like.

The term “neutral ligand” refers to a substituent without a charge, capable of coordination with a metallic center (ruthenium atom). Examples of such ligands can be: amines, phosphines and their oxides, phosphorines and alkyl and aryl phosphates, arsins and their oxides, ethers, alkyl and aryl sulfides, coordinated hydrocarbons, alkyl and aryl halides. In this patent application, the neutral ligand should also be understood as the N-heterocyclic ligand (NHC) and the carbene cycloalkylamine ligand (CAAC).

The term “anionic ligand” refers to a substituent capable of coordination with a metallic center (ruthenium atom) endowed with a charge capable of partially or completely compensating for the charge of the metallic center. Examples of such ligands can be fluoride, chloride, bromide, iodide, cyanide, cyanate and thiocyanate anions, carboxylic acid anions, alcohol anions, phenol anions, thiol and thiophenol anions, hydrocarbon anions with a delocalized charge (e.g. cyclopentadiene), anions of (organo)sulfuric and (organo)phosphoric acids and their esters (such as e.g. anions of alkylsulfonic and arylsulfonic acids, anions of alkylphosphoric and arylphosphoric acids, anions of alkyl esters and aryl sulphuric acid, anions of alkyl esters and aryl phosphoric acids, anions of alkyl esters and aryl alkylphosphoric and aryl phosphoric acids). Optionally, the anionic ligand can have groups L¹, L² and L³, combined such as catechol anion, acetylacetone anion, salicylic aldehyde anion. Anionic ligands (X¹, X²) and neutral ligands (L¹, L², L³) can be combined with each other to form multi-dentate ligands, for example, two-dentate ligand (X¹—X²), tri-dentate ligand (X¹—X²-L¹), four-dentate ligand (X¹—X²-L¹-L²), two-dentate ligand (X¹-L¹), tri-dentate ligand (X¹-L¹-L²), four-dentate ligand (X¹-L¹-L²-L³), two-dentate ligand (L¹-L²), three-dentate ligand (L¹-L²-L³. Examples of such ligands are: catechol anion, acetylacetone anion and salicylaldehyde anion.

The term “heteroatom” means an atom selected from a group that includes oxygen, sulfur, nitrogen, phosphorus, and others.

### EXAMPLES OF INVENTION

The following examples are provided only to illustrate the invention and to clarify its various aspects, not to limit it, and should not be equated with its entire scope, which is defined in the attached claims. In the following examples, unless otherwise indicated, standard materials and methods used in the field of invention were used or the recommendations of the manufacturers of specific reactants and devices were followed, and the use of methods known in the literature of the subject.

Diethyl malonate (S1), methyl Z-oleate, nG-SIPr, nG-SIPr—I₂, UltraCat and UltraNitroCat-I₂ are commercially available compounds. The Phenoxy-Cl complex was obtained according to the literature method [H. Plenio et al., Adv. Synth. Catal., 2013, 355, 439-447]. S1 and methyl oleate were distilled from above activated-aluminium oxide under reduced pressure and stored over activated aluminium oxide. Pure Z-5-deken (99% Z isomer) was obtained by the literature method [A. Hoveyd et al., Organometallics 2011, 30, 1780-1782], then distilled from above activated aluminium oxide and stored over activated aluminium oxide. Other commercially available reagents were used without further purification. All reactions were carried out in an argon atmosphere. Toluene was washed with citric acid, water, dried with 4 Å molecular sieves and deoxidized with argon.

The composition of reaction mixtures was tested by gas chromatography using the PerkinElmer Clarus 680 GC apparatus equipped with the GL Sciences InertCap® 5MS/NP capillary column.

The individual components of the reaction mixtures were identified by comparing retention times with commercial or isolated standards from reaction mixtures for which the structure was confirmed by NMR.

### Example I

### Reaction of Obtaining (Pre)Catalyst Ble-2′

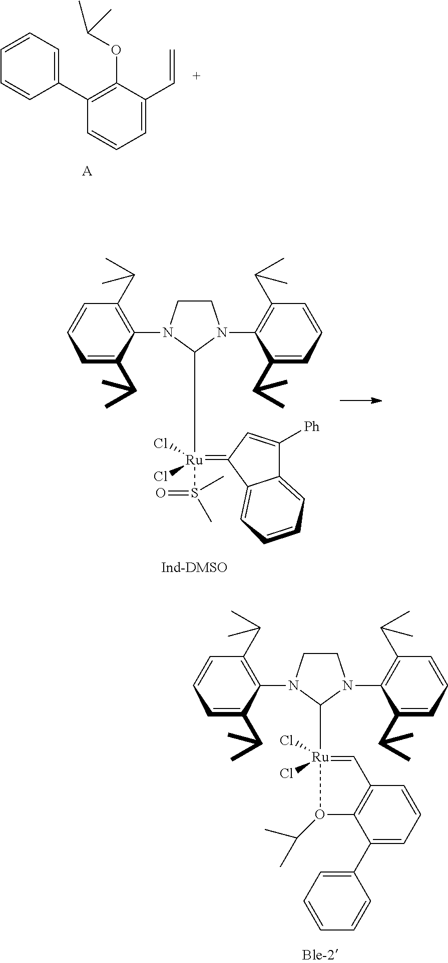

Ligand A [Angew. Chem. Int. Ed. 2002, 41, 2509-2511] and the Ind-DMSO complex were obtained according to literature methods [WO2018038928A1].

The Ind-DMSO ruthenium complex (1.22 g, 1.47 mmol) was dissolved under an argon atmosphere in 12 mL of dichloromethane, followed by the addition of ligand A (0.70 g, 2.94 mmol, 2 equivalents). Stirred at boiling point for 2 hours was performed. Then the solvent was removed, and the remaining mixture was dried under reduced pressure. The raw product was applied to a chromatographic column in a small amount of dichloromethane. The mixture was chromatographically purified with an eluent (cyclohexane/ethyl acetate in a volumetric proportion of 9/1). Solvents were removed, green amorphous solid was mixed for 10 minutes with n-pentane (15 mL) and then cooled to 4° C. Filtered and washed with cold n-pentane. The product was crystallized from a DCM/MeOH mixture. A green, crystalline solid Ble-2′ (0.75 g, 65%) was obtained.

¹H NMR (CD₂Cl₂, 600 MHz) ppm: 16.55 (s, 1H); 7.59-7.56 (m, 2H); 7.48-7.46 (m, 2H); 7.43-7.35 (m, 8H); 6.99-6.96 (m, 1H); 6.87-6.86 (m, 1H); 4.45 (sept, 1H, J=6.6 Hz); 4.23 (s, 4H); 3.67-3.64 (m, 4H); 1.29 (d, 24H, J=6.6H); 0.93 (d, 6H, J=6.6 Hz).

### Example II

Obtaining of Ligand F, in the Reaction Sequence: (i) Alkylation, (ii) Claisen Rearrangement, (iii) Alkylation, (iv) C═C Bond Migration

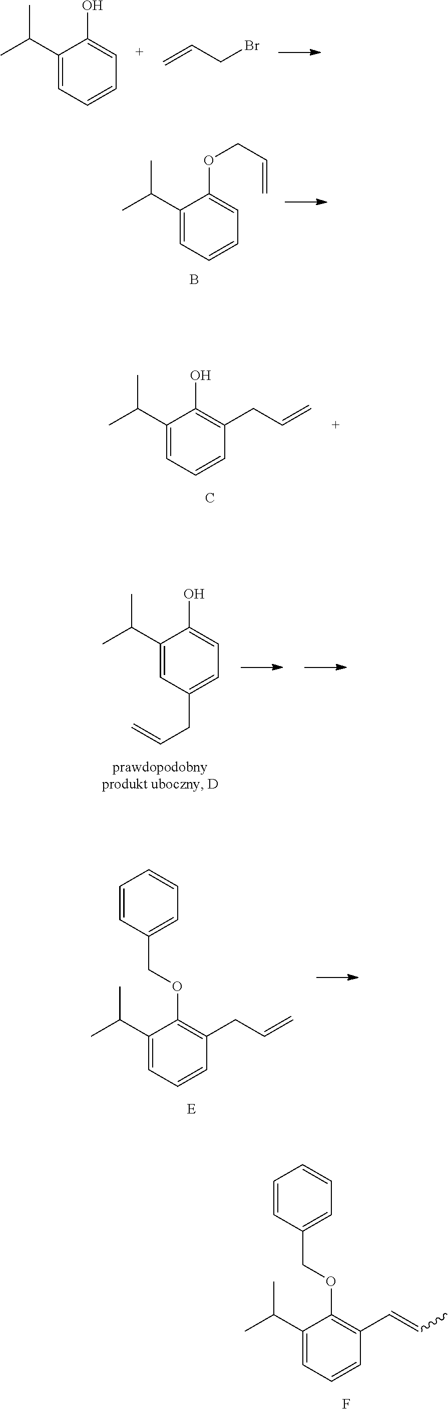

Potassium carbonate (1983 g, 1.4 equivalent), cesium carbonate (166 g, 0.05 equivalent) and allyl bromide (1063 ml, 1.2 equivalent) were added to the vigorously mixed solution of 2-isopropylphenol (1,1396 g, 1396 g, 1.3 mole, 1 equivalent) in acetonitrile (5.9 L [at C_{2-isopropylphenol}=1.5 mola]). The resulting suspension was stirred at boiling point for 22 hours, then the mixture was concentrated to about 50% of the original volume and cooled to ambient temperature. TBME (3 L) was then added to precipitate inorganic by-products, which were filtered, flushed with TBME (1 L) and discarded. The combined extracts of acetonitrile and MTBE were evaporated, after which the raw product (B) was purified by distillation (oil bath temperature 94-96° C., p=5×10⁻² mbar). The first (20 g) and last fraction (60 g) were rejected. Yield: 1680 g (93%). Purity (based on GC): 98%. A round-bottomed 4 L double-necked flask containing 2-isopropylyloxyphenol B (2,1640 g) under argon atmosphere was placed in a heated Radley dish heated to 250° C. Accurate temperature measurements were made with a thermometer immersed in a reaction mixture. The reaction mixture began to boil at 196° C. and as a result of the reaction progressed it reached a boiling point of 216° C. The reaction was continued for 2 hours. Then the raw product C was purified by distillation (oil bath temperature 104-106° C., p=1.3-1.7×10⁻² mbar). Colorless oil, yield: 1200 g (73%). Purity (based on GC): 95%.

Potassium carbonate (1317 g, 1.4 equivalent), cesium carbonate (111 g, 0.05 equivalent) and benzyl bromide (890 ml, 1.1 equivalent) were added successively to a vigorously mixed solution of 2-allyl-6-isopropylphenol (C) (3,1200 g, 6.8 mol, 1 equivalent) in acetonitrile (ACN=3,65 1 [C_{2-allyl-6-isopropylphenol}=1.5 mol]). The resulting suspension was heated at boiling point for 16 hours, then concentrated to about 50% of the original volume and cooled to ambient temperature. TBME (3 L) was then added to precipitate inorganic by-products, which were filtered, washed with TBME (1 L) and discarded. The solvents were evaporated on a rotary evaporator at 80° C. (residual benzyl bromide was partially removed). The raw product (E, 1800 g, 99%, yellow oil) was used in the next stage without further purification.

Carbonylchlorohydridotris(triphenylphosphine)ruthenium (32.2 g, 0.5 mol %) was added in 10 portions to a boiling solution of 2-allyl-6-isopropylbenzybenzene (E) (5,1800 g, 6.76 mol, 1 equivalent) in toluene (8.45 L, C=0.8 M) in 1 hour. Stirring was continued until the temperature of the reaction mixture returned to the ambient temperature. Then the post-reaction mixture was filtered through 500 g of silica gel. Toluene was removed on a rotary evaporator and the raw product F was purified by distillation (heating bowl temperature 160-170° C., p=7×10⁻² mbar), collecting light yellow oil. Efficiency: 1567 g, (yield 87%, E/Z=8/2). Purity (based on GC): 98%.

### Example III

Reaction of Obtaining (Pre)Catalyst 1a-Cl₂

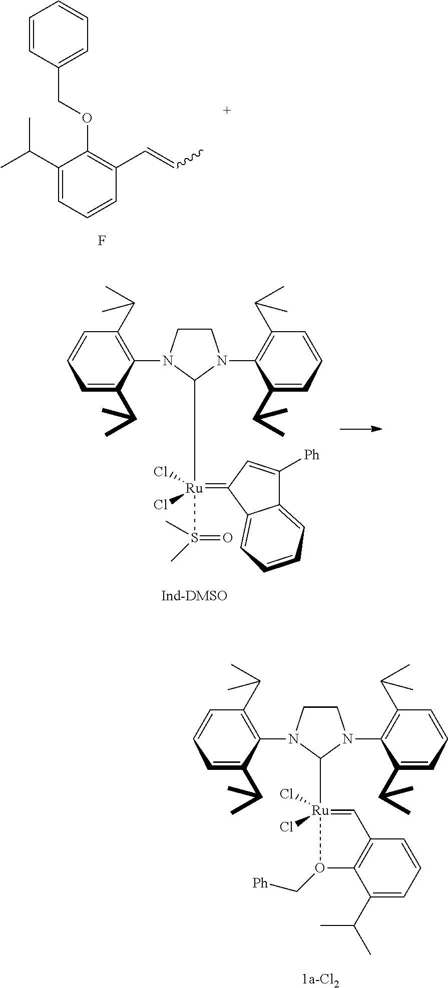

The Ind-DMSO complex (1.0 g, 1.20 mmol) in the argon atmosphere was dissolved in 10 mL of dichloromethane, and then the ligand F (0.417 g, 1.56 mmol, 1.3 equivalent) was added. Then the reaction mixture obtained in this way was stirred at boiling point for 2 hours. The solvent was removed, and the residue was dried under reduced pressure. The raw product was crystallized twice from a mixture of DCM/MeOH solvents. A green crystalline solid of 1a-Cl₂ (0.76 g, yield of 79%) was obtained.

¹H NMR (CD₂Cl₂, 600 MHz) ppm: 16.56 (s, 1H); 7.59-7.56 (m, 2H); 7.51-7.50 (m, 1H); 7.37-7.36 (m, 4H); 7.34-7.27 (m, 5H); 7.02 (t, 1H, J=7.2 Hz); 6.75-6.74 (m, 1H); 5.29 (s, 2H); 4.21 (s, 4H); 3.60-3.55 (m, 4H); 3.16 (sept, 1H, J=6.6 Hz); 1.27 (d, 12H, J=7.2H); 1.16 (d, 6H, J=6.6 Hz); 1.12 (d, 12H, J=6.0 Hz).

### Example IV

Reaction of obtaining (pre)catalyst 1b-Cl₂ and 1b-I₂

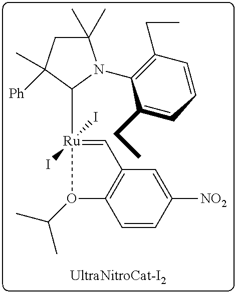

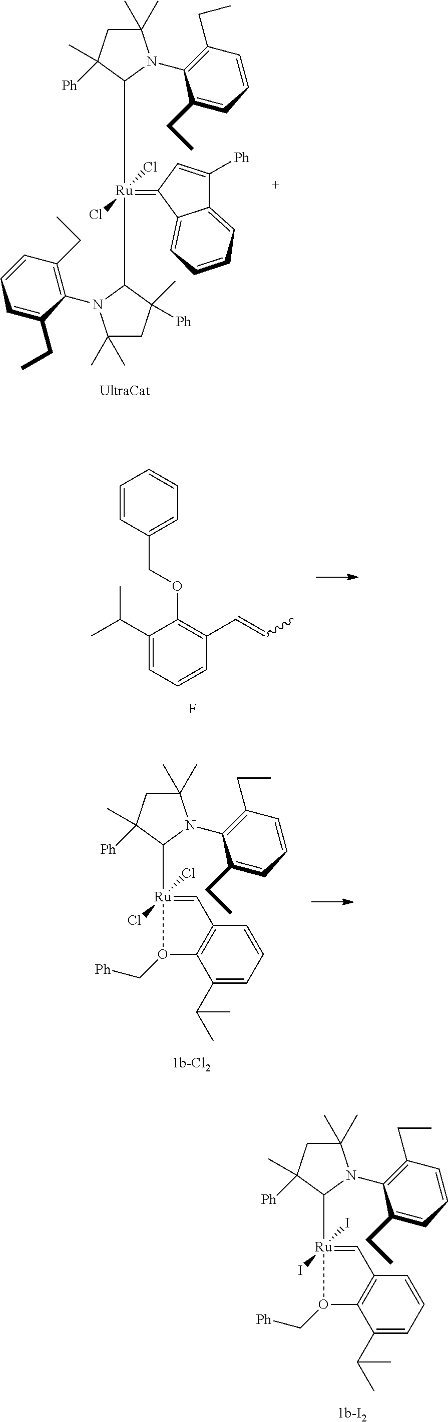

To the solution of the UltraCat complex (1.0 g, 1.04 mmol) in toluene (7 mL) ligand F was added (0.33 g, 1.25 mmol, 1.25 mmol, 1.2 equivalent) and copper chloride (I) (0.155 g, 1.56 mmol, 1.5 equivalent). The reaction mixture was heated to 70° C. After 1 hour, the reaction mixture was cooled to ambient temperature and filtered through a thin layer of celite. Toluene was removed on a rotary evaporator. The residue was crystallized twice from the DCM/MeOH mixture. The resulting green solid (1b-Cl₂) was dried under a high vacuum, then dissolved in acetone (15 mL) and sodium iodide (1.56 g, 10.43 mmol) was added. Then the whole was stirred at 40° C. for 3 hours. Acetone was removed and DCM was added to the residue. Filtered through Schott's funnel. The filtrate was evaporated and reconstituted in acetone (15 mL). Sodium iodide (1.56 g, 10.43 mmol) was added and stirred at 40° C. for 6 hours. Acetone was removed and DCM was added to the residue. Filtered through Schott's funnel. The filtrate was evaporated. The product (1b-I₂) was crystallized from a DCM/MeOH mixture to obtain a dark green crystalline solid (0.486 g, with a yield of 51%).

### Example V

### Comparison of Reactivity of (Pre)Catalysts in the RCM Model Reaction

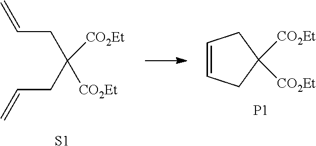

The solution of diethyl malonate (S1) in toluene (10 mL, 0.1 M) was cooled to 0° C. and then a solution of (pre)catalyst (Ble-2, 1a-Cl₂, or nG-SiPr at 0.1% mol) in toluene (100 μL) was added. During the entire reaction, argon was passed through the reaction mixture to actively remove the ethylene emitted. At appropriate intervals of time, 0.2 mL of reaction mixture was taken and transferred to a vial containing 0.8 mL of ethyl-vinyl ether. The samples were analyzed using a gas chromatograph.

TABLE 1
Comparison of catalyst activity in the model RCM reaction

| | Ble-2 | | 1a-Cl₂ | | nG-SiPr | |
|---|---|---|---|---|---|---|
| t[min] | Q1 | S1 | Q1 | S1 | Q1 | S1 |
| 2 | 6.7 | 92.5 | 6.9 | 92.5 | 1.7 | 97.6 |
| 4 | 12.4 | 87.0 | 15.1 | 84.2 | 1.9 | 97.4 |
| 6 | 20.4 | 78.8 | 22.6 | 76.7 | 2.3 | 97.0 |
| 8 | 28.7 | 70.7 | 31.3 | 67.9 | 3.0 | 96.3 |
| 10 | 36.5 | 62.8 | 39.6 | 59.7 | 3.4 | 95.9 |
| 20 | 63.3 | 36.1 | 67.1 | 32.3 | 7.3 | 92.0 |
| 30 | 74.3 | 25.0 | 75.3 | 24.0 | 12.5 | 86.8 |
| 60 | 85.2 | 14.1 | 84.9 | 14.4 | 26.7 | 72.6 |

### Example VI

Reaction of Obtaining (Pre)Catalyst 1a-S₂

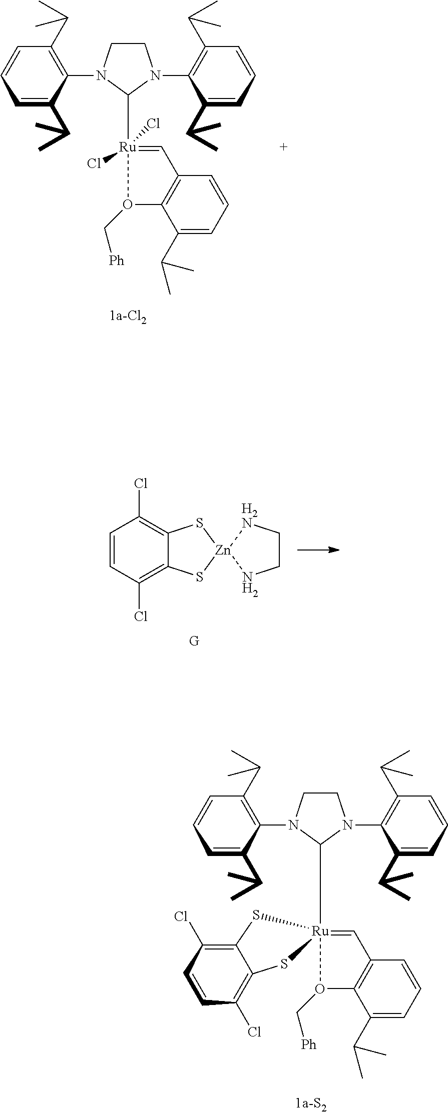

The entire reaction and isolation of the product was carried out in anaerobic conditions, under argon. DCM (1500 ml) was degassed with argon. The degassed DCM was supplemented with the 1a-Cl₂ complex (250 g, 312 mmol) and the ligand G (125 g, 374 mmol, 1.2 equivalent). The whole was mixed for 60 minutes at ambient temperature. DCM (1500 ml) was degassed in a separate flask. With the help of a cannula, the reaction mixture was transferred to the sinter and filtered without air access to the 5 L three-necked flask with two caps with tap. One of the caps was connected to the argon line, and the other to the evaporator. The flask was rinsed after the reaction with previously degassed DCM. The sintered sludge was washed with degassed methanol (1 L). At the end of the filtration, there were 3 L of DCM and 1 L of MeOH in the flask. The flask was placed in a heating bowl heated to 40° C. and the DCM was slowly removed. After evaporation of DCM, the flask was filled with argon and the product was scraped off the walls with a spatula. Then, stirring, with the help of a cannula, the suspension was transferred to sinter and methanol was filtered. The sludge was washed with degassed methanol until the filtrate was light yellow, then the sludge was washed with degassed n-pentane until the filtrate was light yellow. Product 1a-S₂ was transferred to the flask and dried under reduced pressure. Process efficiency 278 g, (95% yield) of the expected product 1a-S₂ obtained in the form of brownish-yellow powder.

A spectrum of ¹H NMR was made in a degassed CD₂Cl₂. The disappearance of the characteristic signal from the benzylidene proton with a displacement of 16.56 ppm and the appearance of a new signal from the benzylidene proton with a displacement of 14.52 ppm were observed.

### Example VII

Reaction of Obtaining (Pre)Catalyst 1e-S₂

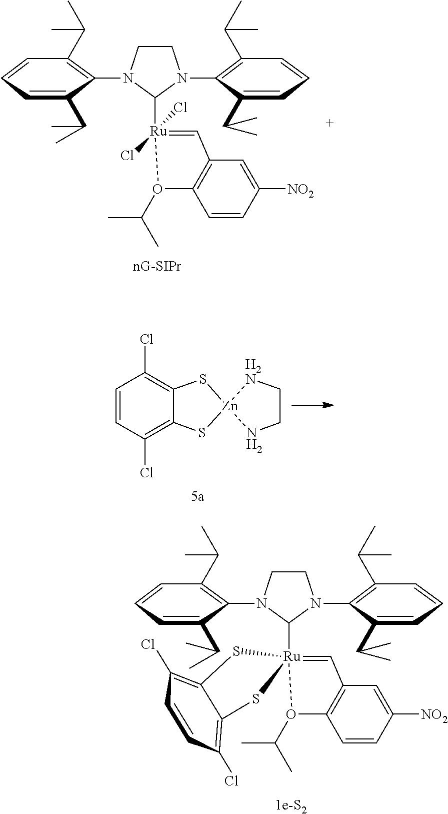

The 1e-S₂ complex was obtained according to the procedure described in example VI.

In this way, 1.4 g of the target product 1e-S₂ with the yield of 99% was obtained.

A spectrum of ¹H NMR was made in a degassed CD₂Cl₂. The disappearance of the characteristic signal from the benzylidene proton with a displacement of 15.43 ppm and the appearance of a new signal from the benzylidene proton with a displacement of 14.41 ppm were observed.

### Example VIII

Reaction of Obtaining (Pre)Catalyst 1d-S₂

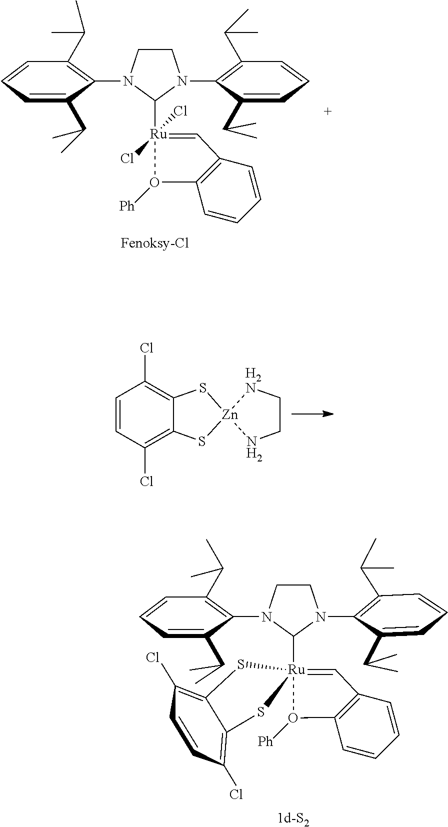

The 1d-S₂ complex was obtained according to the procedure described in example VI. In this way, 0.3 g of 1d-S₂ product with the yield of 99% was obtained.

A spectrum of ¹H NMR was made in a degassed CD₂Cl₂. The disappearance of the characteristic signal from the benzylidene proton with a displacement of 16.56 ppm and the appearance of a new signal from the benzylidene proton with a displacement of 14.48 ppm were observed.

### Example IX

Comparison of the effectiveness of complexes 1a-S₂, 1d-S₂, 1e-S₂ in stereoretentive cross metathesis.

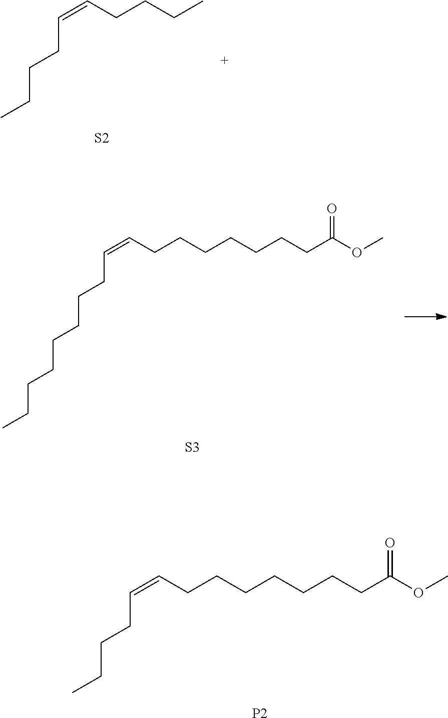

The Z-5-dekene (3.0 mmol, 0,577 mL), methyl Z-oleate MO (1.0 mmol, 0,340 mL) and 0.07 mL hexadecane (internal standard) were placed in a round-bottomed flask under an argon atmosphere and heated to 45° C. Suitable complexes (1e-S₂, 1a-S₂, 1d-S₂) were added in 5 ppm molar portions for a double bond of C═C. After each portion, the reaction was carried out for 30 minutes, after which time a sample was taken for GC analysis. The results are presented in the table below. In each case, products with a Z/E isomer ratio of 99/1 were obtained.

TABLE 2
Comparison of the efficiency of stereoretentive catalysts.

| | | | Loading | Conversion |
|---|---|---|---|---|
| No | (Pre)cat. | Time (min) | (ppm/C═C) | (%) |
| 1 | 1e-S₂ | 30 | 5 | 7.0 ± 2.2 |
| 2 | 1a-S₂ | 30 | 5 | 7.0 ± 3.0 |
| 3 | 1d-S₂ | 30 | 5 | 10.5 ± 0.2 |
| 4 | 1e-S₂ | 60 | 10 | 22.5 ± 2.5 |
| 5 | 1a-S₂ | 60 | 10 | 42.0 ± 5.0 |
| 6 | 1d-S₂ | 60 | 10 | 43.2 ± 5.3 |
| 3 | 1e-S₂ | 90 | 15 | 43.0 ± 3.6 |
| 6 | 1a-S₂ | 90 | 15 | 82. ± 2.6 |
| 9 | 1d-S₂ | 90 | 15 | 50.7 ± 0.3 |

1. A ruthenium complex selected from the group consisting of the structures 1a-Cl₂, 1a-I₂, 1c-Cl₂, and 1c-I₂:

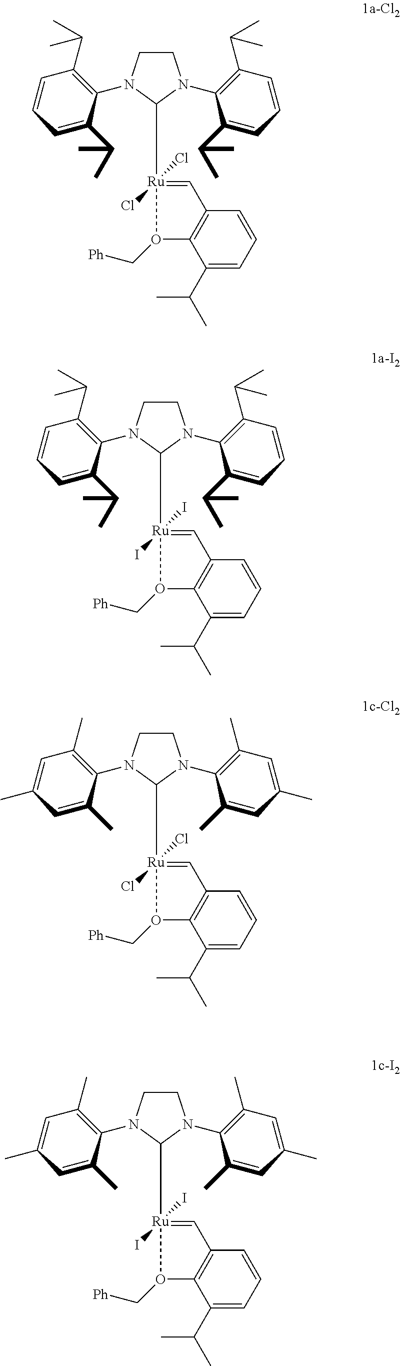

2. A method of reacting in an olefin metathesis reaction a compound according to claim 1 as a precatalyst or catalyst in the olefin metathesis reaction.

3. The method according to claim 2, wherein the reaction is carried out in an organic solvent, selected from toluene, benzene, mesitylene, dichloromethane, ethyl acetate, methyl acetate, tertbutyl-methyl ether, cyclopentyl-methyl ether or in a solvent-free system

4. The method according to claim 2, wherein the reaction is carried out at a temperature of 20 to 150° C. and in a time from 5 minutes to 24 hours, wherein compound according to claim 1 is used in an amount of not more than 0.1% molar and wherein compound according to claim 1 is added to the reaction mixture in solid or continuously by using vacuum pump as a solution and wherein the gaseous by-product of the reaction selected from ethylene, propylene and butylene is actively removed from the reaction mixture by inert gas barbotage or by the vacuum.

5. The method according to claim 3, wherein the reaction is carried out at a temperature of 20 to 150° C. and in a time from 5 minutes to 24 hours, wherein compound according to claim 1 is used in an amount of not more than 0.1% molar and wherein compound according to claim 1 is added to the reaction mixture in solid or continuously by using vacuum pump as a solution and wherein the gaseous by-product of the reaction selected from ethylene, propylene and butylene is actively removed from the reaction mixture by inert gas barbotage or by the vacuum.

6. The method of claim 2, wherein the olefin metathesis reaction is selected from the group consisting of a ring closing metathesis (RCM), a homometatesis, a cross metathesis (CM), an ethenolysis, an isomerization, a diastereoselective ring rearrangement metathesis (DRRM) reaction, an alken-alkin metathesis (en-yn) and a ring-opening metathesis polymerization (ROMP) reaction.
